(19) United States
(12) Patent Application Publication
(10) Pub. No.: US 2024/0342393 A1
(43) Pub. Date: Oct. 17, 2024

(54) NEEDLE AND NEEDLE TIP INCLUDING THE SAME

(71) Applicants: Suk Bae SEO, Seoul (KR); Ja Young KIM, Seoul (KR); Ho Joon SEO, Seoul (KR)

(72) Inventors: Suk Bae SEO, Seoul (KR); Ja Young KIM, Seoul (KR); Ho Joon SEO, Seoul (KR)

(21) Appl. No.: 18/751,656

(22) Filed: Jun. 24, 2024

Related U.S. Application Data
(63) Continuation of application No. PCT/KR2022/021474, filed on Dec. 28, 2022.

(30) Foreign Application Priority Data
Jan. 4, 2022 (KR) 10-2022-0000861

(51) Int. Cl. A61M 5/32

(52) U.S. Cl. CPC A61M 5/3286; A61M 2205/82

(57) ABSTRACT

Disclosed are a needle that may inject a drug in communication with a targeted depth of a target while minimizing a depth, by which the needle is inserted into the target when the drug is injected to the targeted depth of the target through the needle, whereby an unnecessary injury may be prevented from occurring as an unnecessary portion is inserted into the target, and a needle tip including the same.

### CROSS-REFERENCE TO RELATED APPLICATIONS

The present application is a continuation of International Patent Application No. PCT/KR2022/021474, filed on Dec. 28, 2022, which is based upon and claims the benefit of priority to Korean Patent Application No. 10-2022-0000861 filed on Jan. 4, 2022. The disclosures of the above-listed applications are hereby incorporated by reference herein in their entirety.

### BACKGROUND

Embodiments of the present disclosure described herein relate to a needle and a needle tip including the same.

In general, a method of penetrating a drug into skin is used as a treatment method for skin beauty and health, such as removal of wrinkles, removal of acne, exfoliation, and treatment of hair loss, and treatment of obesity.

When a drug is delivered by applying it to the skin, it is difficult for the drug to penetrate the stratum corneum, so a skin absorption rate is very low. To improve this, a technology of inserting a needle into the skin and then injecting a drug into the skin through the needle has been developed and is currently being widely used.

Furthermore, recently, a method of creating microwounds for promoting regeneration of skin from epithelium to dermis by inserting a needle into the skin and then providing radio frequencies (RFs) to the skin through the needle to apply a local thermal damage to the skin and causing the microwounds to induce cell growth factors to maximize natural healing and regeneration of the skin.

FIG. 1 is a schematic view illustrating a state, in which a drug is injected into skin through a conventional needle.

As illustrated in FIG. 1, the conventional needle has a single inclined part in the form of an inclined cut.

When a drug is injected into a targeted depth of the skin through the conventional needle 1, the needle 1 has to be inserted to a depth L1 that is deeper than a targeted depth 12 of skin 10 so that there is no other skin tissue that hampers injection of the drug between a tip end portion of the needle 1 and the targeted depth 12 of the skin 10, and thus the tip end portion of the needle 1 communicates to the targeted depth 12 of the skin 10 to inject the drug whereby an unnecessary injury occurs to the skin.

### SUMMARY

Embodiments of the present disclosure provide a needle that may inject a drug in communication with a targeted depth of a target while minimizing a depth, by which the needle is inserted into the target when the drug is injected to the targeted depth of the target through the needle, whereby an unnecessary injury may be prevented from occurring as an unnecessary portion is inserted into the target, and a needle tip including the same.

The problems to be solved by the present disclosure are not limited to the problems mentioned above, and other problems not mentioned can be clearly understood by those skilled in the art from the description below.

According to an aspect of the present disclosure, a needle includes a needle body having a specific length, defining a drug passage, through which a drug flows, in an interior thereof, and into which a target, into which the drug is injected, is inserted, a cutting part disposed in the needle body across the drug passage, and that cuts the target, and a pair of inclined parts cut to be inclined along a lengthwise direction of the needle body with respect to a central axis line of the needle body at a tip end portion of the needle body while the cutting part being interposed therebetween, and defining a pair of drug discharge holes communicating with the drug passage.

Furthermore, the pair of drug discharge holes may be formed to be symmetrical to each other with respect to the central axis line of the needle body.

Furthermore, the cutting part may be disposed at the tip end portion of the needle body.

According to another aspect of the present disclosure, a needle tip includes the at least one needle, and a needle fixing part, to which the one or more needles are fixed, on one surface thereof.

Furthermore, the needle tip may further include a needle adapter provided between the needle and the needle fixing part, and connecting the needle and the needle fixing part, and a needle cap having a pass hole, through which the needle passes, is formed, at a tip end portion thereof, having a coupling part coupled to the needle adapter at a rear end portion thereof, that adjusts a length, by which the needle is exposed to an outside of the pass hole, depending on a location, at which the coupling part is coupled to the needle adapter.

Furthermore, the needle cap may be screw-coupled to the needle adapter.

Furthermore, the needle tip may further include a scale display part displayed on the needle cap by scales along a direction, in which the needle is exposed to the outside of the pass hole.

Furthermore, the needle tip may further include a power supply part provided in the needle fixing part, and that selectively applies current to the needle.

The other detailed items of the present disclosure are included in a detailed description and the drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic view illustrating a state, in which a drug is injected into skin through a conventional needle;

FIG. 2 is a perspective view illustrating a needle according to an embodiment of the present disclosure;

FIG. 3 is an exploded perspective view illustrating a needle according to an embodiment of the present disclosure;

FIG. 4 is a schematic view illustrating a state, in which a drug is injected into skin through a needle according to an embodiment of the present disclosure;

FIG. 5 is a perspective view illustrating a needle tip according to an embodiment of the present disclosure;

FIGS. 6 and 7 are perspective views illustrating a state, in which a length, by which a needle is exposed at a needle tip, is adjusted according to an embodiment of the present disclosure; and

FIG. 8 is a schematic view illustrating a state, in which a needle tip is mounted on a skin treatment device according to an embodiment of the present disclosure.

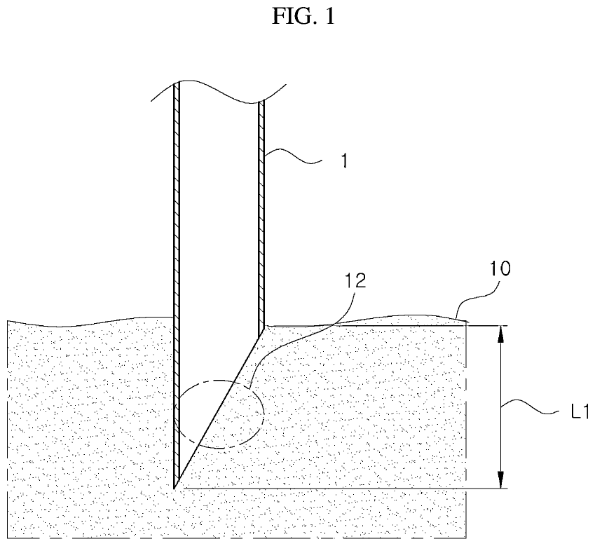

### DETAILED DESCRIPTION

The advantages and features of the present disclosure, and a method for achieving them will become clear with reference to the embodiments that will be described in detail together with the accompanying drawings. However, the present disclosure is not limited by the embodiments disclosed hereinafter but may be implemented in various different forms, and the embodiments are provided simply to make the disclosure of the present disclosure complete and inform an ordinary person in the art of the scope of the present disclosure, and the disclosure is only defined by the scope of the claims.

Unless otherwise defined, all terms (including technical and scientific terms) used herein have the same meaning as commonly understood by those skilled in the art, to which the present disclosure pertains. Furthermore, the terms defined in commonly used dictionaries should not be interpreted in an idealized or overly formal sense unless expressly so defined herein.

Hereinafter, embodiments of the present disclosure will be described in detail with reference to the accompanying drawings.

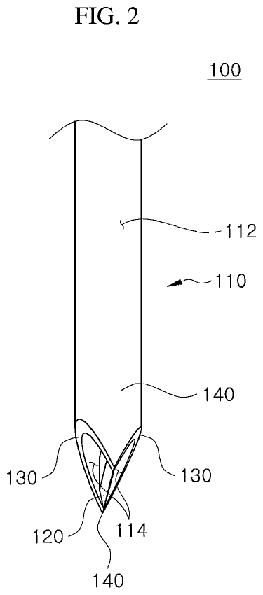

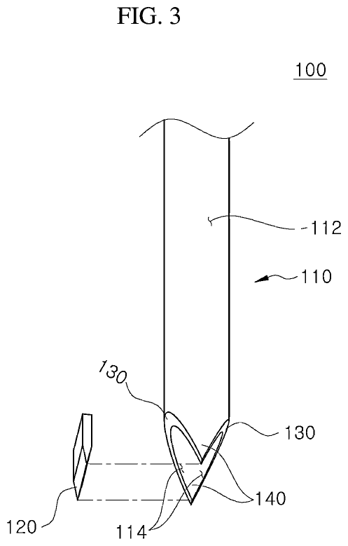

FIG. 2 is a perspective view illustrating a needle according to an embodiment of the present disclosure, and FIG. 3 is an exploded perspective view illustrating the needle according to an embodiment of the present disclosure.

As illustrated in FIGS. 2 and 3, a needle 100 according to an embodiment of the present disclosure may include a needle body 110, a cutting part 120, and a pair of inclined parts 130.

The needle body 110 may have a specific length, and may define a drug passage 112, through which a drug flows, and a target 10, into which the drug is injected, may be inserted thereinto. For example, the needle body 110 may be inserted from a surface of the target 10 to a targeted depth 12 of the target 10 and then the drug may be injected to the targeted depth 12 of the target 10 through the drug passage 112. Here, the target 10 is not particularly limited, but may be the skin of the patient.

The needle body 110 may be formed in a cylindrical tube shape to facilitate insertion of the target 10. That is, the needle body 110 may have a shape having an opened distal end portion. Furthermore, the needle body 110 may receive the drug from a syringe body, or may receive the drug from a needle fixing part 200 that will be described later.

The needle body 110 may have a cannula shape having a closed distal end portion, and may be manufactured by cutting the cannula-shaped distal end portion, and the cutting part 120 that will be described later may be disposed at the cut distal end portion.

The cutting part 120 may be disposed in the needle body 110 across the drug passage 112 to cut the target 10.

The cutting part 120 is not particularly limited, but may have a sharp edge shape along a central axis line of the needle body 110. Here, the cutting part 120 is not particularly limited, but may have a rectangular shape that facilitates cutting of the target 10.

The cutting part 120 may be fitted or welded between the tip end portions of a pair of division parts 140 that will be described later.

The cutting part 120 may be disposed at the tip end portion of the needle body 110. In this example, when the needle body 110 is inserted from the surface of the target 10 to the targeted depth 12 of the target 10, an insertion path of the target 10 of the needle body 110 may be secured while the cutting part 120 cuts a portion of a target 120, which contacts the tip end portion of the needle body 110, along a depth direction of the target 10.

As a result, even when the tip end portion of the needle body 110 is inserted only up to the targeted depth 12 of the target 10, the tip end portion of the needle body 110 communicates with the targeted depth 12 of the target 10, and thus, the drug may be injected to the targeted depth 12 of the target 10 through the drug passage 112 of the needle body 110. Accordingly, because a depth L2, by which the needle 100 is inserted into the target 10, is minimized and the needle 100 communicates with the targeted depth 12 of the target 10, an unnecessary injury in the target 10 may be prevented.

The cutting part 120 may be disposed to protrude toward the target 10 further than the tip end portion of the needle body 110. In this example, when the needle body 110 is inserted from the surface of the target 10 to the targeted depth 12 of the target 10, a portion of the cutting part 120, which faces the tip end portion of the needle body 110, may be preferentially cut along the depth direction of the target 10.

The pair of inclined parts 130 may be cut to be inclined along a lengthwise direction of the needle body 110 with respect to the central axis line of the needle body 110 at the tip end portion of the needle body 110 while the cutting part 120 being interposed therebetween to form a pair of drug discharge holes 114 that communicate with the drug passage 112.

The pair of inclined parts 130 may have a shape, in which a distance from the central axis line of the needle body 110 becomes smaller as it goes in a direction that becomes closer to the tip end portion of the needle body 110.

A pair of division parts 140 having a “V” shaped cross-sectional shape may be provided between the pair of inclined parts 130, and the cutting part 120 may be provided between the tip end portions of the pair of division parts 140. Accordingly, even though only the tip end portions of the pair of division parts 140 are inserted to the targeted depth 12 of the target 10 when the needle body 110 is inserted from the surface of the target 10 to the targeted depth 12 of the target 10, the drug may be injected to the targeted depth 12 of the target 10 through the drug passage 112 of the needle body 110. Meanwhile, the pair of division parts 140 may define a boundary between the pair of inclined parts 130.

The pair of drug discharge holes 114 may be formed to be symmetrical to each other with respect to the central axis line of the needle body 110.

Hereinafter, a process of injecting a drug into skin through the needle according to an embodiment of the present disclosure will be described.

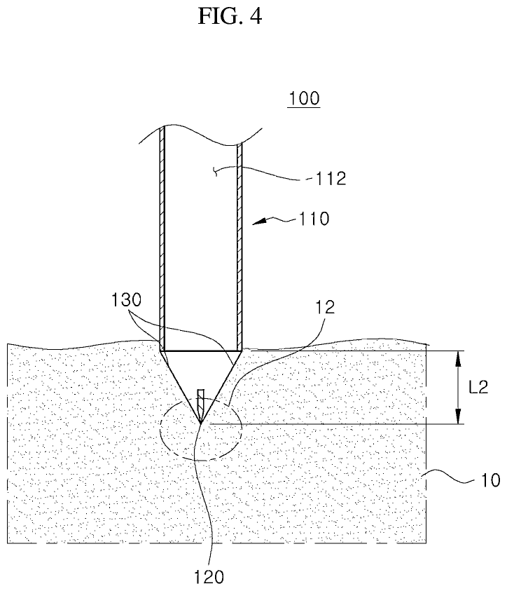

FIG. 4 is a schematic view illustrating a state, in which a drug is injected into skin through a needle according to an embodiment of the present disclosure.

As illustrated in FIG. 4, first, the needle body 110 is inserted from the surface of the target 10 to the target depth of the target 10. Then, the cutting part 120 cuts the portion of the target 10, which faces the tip end portion of the needle body 110, along the depth direction of the target 10, the insertion path of the target 10 of the needle body 110 may be secured, and thus, an insertion efficiency of the needle body 110 may be improved.

Next, the drug is injected to the targeted depth 12 of the target 10 through the drug passage 112 of the needle body 110. Then, the needle body 110 may inject the drug supplied through the drug passage 112 from the syringe body to the targeted depth 12 of the target 10, or may inject the drug supplied from the needle fixing part 200 that will be described later to the drug passage 112 to the targeted depth 12 of the target 10.

Accordingly, according to the needle 100 according to an embodiment of the present disclosure, because a depth L2, by which the needle 100 is inserted into the target 10, is minimized and the needle 100 communicates with the targeted depth 12 of the target 10 to inject the drug, an unnecessary injury in the target 10 may be prevented.

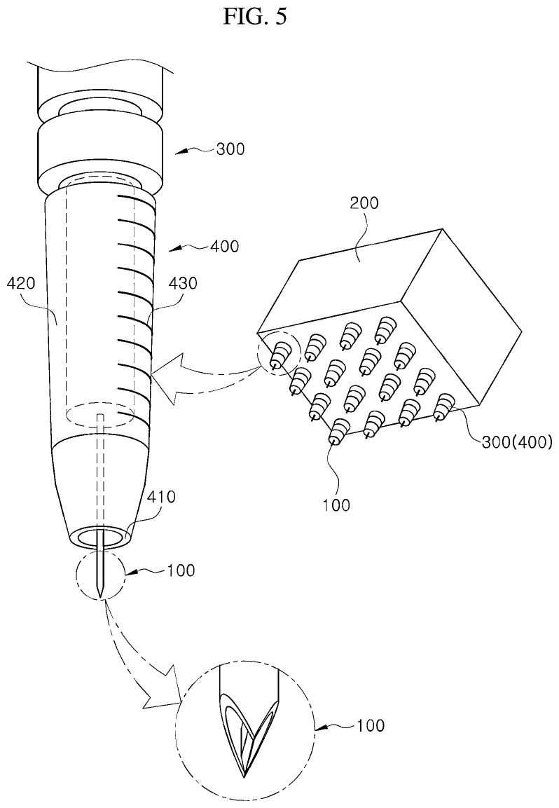

FIG. 5 is a perspective view illustrating a needle tip according to an embodiment of the present disclosure.

As illustrated in FIG. 5, a needle tip according to an embodiment of the present disclosure may include one or more needles 100 and a needle fixing part 200. Here, the needle 100 has already been described, and thus, a detailed description thereof will be omitted.

The needle fixing part 200 serves to fix the one or more needles 100. The one or more needles 100 may be fixed to a surface of the needle fixing part 200.

The needle fixing part 200 may be provided with a power supply part that selectively applies current to the needle 100. When current is applied to the needle 100 by the power supply part (not illustrated), electrical energy may be transmitted to a targeted portion of the target 10 through the needle 100.

Meanwhile, the needle tip according to an embodiment of the present disclosure may further include a needle adapter 300 and a needle cap 400.

The needle adapter 300 is provided between the needle 100 and the needle fixing part 200, and connects the needle 100 and the needle fixing part 200. In detail, the tip end portion of the needle adapter 300 may be coupled to the needle 100, and a rear end portion of the needle adapter 300 may be coupled to the needle fixing part 200.

The needle adapter 300 is not particularly limited, but may have a shape corresponding to a hub of the needle 100 of the syringe.

In the needle cap 400, a pass hole 410, through which the needle 100 passes, may be formed at the tip end portion, a coupling part 420 that is coupled to the needle adapter 300 may be formed at the rear end portion, and a length of the needle 100, which is exposed to an outside of the pass hole 410, may be adjusted depending on a position, in which the coupling part 420 is coupled to the needle adapter 300. Then, because the needle 100 is inserted into the target 10 by the length that is exposed to the outside of the pass hole 410 of the needle cap 400, the depth, by which the needle 100 is inserted into the target 10, may be adjusted by adjusting the length, by which the needle 100 is exposed to the outside of the pass hole 410 of the needle cap 400.

Meanwhile, the needle tip according to an embodiment of the present disclosure may further include a scale display part 430.

The scale display part 430 may be displayed as scales on the needle cap 400 along a direction, in which the needle 100 is exposed to the outside of the pass hole 410. Accordingly, the length of the needle 100 exposed to the outside of the pass hole 410 in the needle cap 400 may be measured through the scale display part 430.

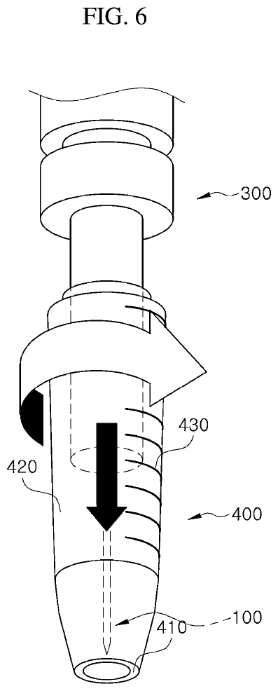

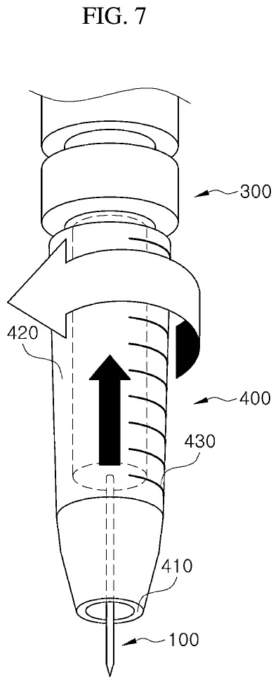

FIGS. 6 and 7 are perspective views illustrating a state, in which a length, by which a needle is exposed at a needle tip, is adjusted according to an embodiment of the present disclosure.

As illustrated in FIGS. 6 and 7, the needle cap 400 may be screw-coupled to the needle adapter 300. For example, a screw thread may be formed on an inner peripheral surface of the needle cap 400, and a screw thread that is screw-coupled to the screw thread of the needle cap 400 may be formed on an outer peripheral surface of the needle adapter 300.

Accordingly, when the needle cap 400 rotates forward and backward, the needle cap 400 may reciprocate along the needle adapter 300, and the length, by which the needle 100 is exposed to the outside of the pass hole 410 of the needle cap 400 may be easily adjusted.

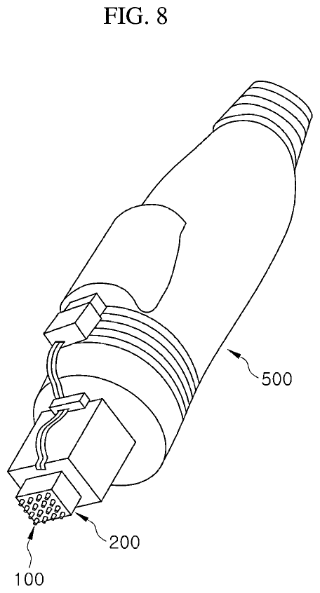

FIG. 8 is a schematic view illustrating a state, in which a needle tip is mounted on a skin treatment device according to an embodiment of the present disclosure.

As illustrated in FIG. 8, a skin treatment device according to an embodiment of the present disclosure may include a handpiece 500, on which a needle fixing part 200, to which the one or more needles 100 are fixed, is mounted.

The handpiece 500 may be provided with a drug supply part (not illustrated) that supplies a drug to the needle 100, and a power supply part (not illustrated) that selectively applies current to the needle 100.

In this example, in a state, in which the user grips the handpiece 500 and inserts the needle 100 to the targeted depth 12 of the target 10, the drug may be supplied from the drug supply part to the drug passage 112 of the needle 100 and the drug is injected to the targeted depth 12 of the target 10, or current may be applied to the needle 100 through the power supply part to supply electrical energy to the targeted depth 12 of the target 10. Here, the target 10 may be skin.

Meanwhile, the handpiece 500 may be provided with a controller (not illustrated) for controlling supply of the drug by the drug supply part and current applied by the power supply part.

According to the present disclosure, the needle and the needle tip including the needle according to an embodiment of the present disclosure may minimize the depth, by which the needle is inserted into the target when the drug is injected to the targeted depth of the target through the needle, and injects the drug while the needle communicates with the targeted depth of the target, and thus, may prevent an unnecessary injury that occurs as an unnecessary portion is inserted into the target.

According to the needle and the needle tip including the same according to an embodiment of the present disclosure, a drug may be injected in communication with the targeted depth of the target while the depth, by which the needle is inserted into the target, is minimized when the drug is injected to the targeted depth of the target through the needle whereby an unnecessary injury may be prevented from occurring as an unnecessary portion is inserted into the target

The effects of the present disclosure are not limited to the effects mentioned above, and other effects not mentioned may be clearly understood by those skilled in the art from the description below.

Until now, the embodiments of the present disclosure have been described with reference to the accompanying drawings, but it may be understood that an ordinary person in the art, to which the present disclosure pertains, may carry out the present disclosure in another specific form while not changing the technical spirit or the essential features. Therefore, it should be understood that the embodiments described above are all illustrative, and not restrictive.

What is claimed is:

1. A needle comprising:
a needle body having a specific length, defining a drug passage, through which a drug flows, in an interior thereof, and into which a target, into which the drug is injected, is inserted;
a cutting part disposed in the needle body across the drug passage, and configured to cut the target; and
a pair of inclined parts cut to be inclined along a lengthwise direction of the needle body with respect to a central axis line of the needle body at a tip end portion of the needle body while the cutting part being interposed therebetween, and defining a pair of drug discharge holes communicating with the drug passage.

2. The needle of claim 1, wherein the pair of drug discharge holes are formed to be symmetrical to each other with respect to the central axis line of the needle body.

3. The needle of claim 1, wherein the cutting part is disposed at the tip end portion of the needle body.

4. A needle tip comprising:
at least one needle claimed in claim 1; and
a needle fixing part, to which the one or more needles are fixed, on one surface thereof.

5. The needle tip of claim 4, further comprising:
a needle adapter provided between the needle and the needle fixing part, and connecting the needle and the needle fixing part; and
a needle cap having a pass hole, through which the needle passes, is formed, at a tip end portion thereof, having a coupling part coupled to the needle adapter at a rear end portion thereof, configured to adjust a length, by which the needle is exposed to an outside of the pass hole, depending on a location, at which the coupling part is coupled to the needle adapter.

6. The needle tip of claim 5, wherein the needle cap is screw-coupled to the needle adapter.

7. The needle tip of claim 5, further comprising:
a scale display part displayed on the needle cap by scales along a direction, in which the needle is exposed to the outside of the pass hole.

8. The needle tip of claim 4, further comprising:
a power supply part provided in the needle fixing part, and configured to selectively apply current to the needle.
